(19) United States
(12) Patent Application Publication
(10) Pub. No.: US 2024/0274296 A1
(43) Pub. Date: Aug. 15, 2024

(54) METHODS OF DETERMINING TIME INTERVAL FOR FURTHER DIAGNOSTICS IN PROSTATE CANCER

(71) Applicant: A3P Biomedical AB, Stockholm (SE)

(72) Inventors: Karl Andersson, Vange (SE); Mats Wallden, Uppsala (SE)

(21) Appl. No.: 18/568,946

(22) PCT Filed: Mar. 25, 2022
(86) PCT No.: PCT/EP2022/057920

§ 371 Date: Dec. 11, 2023

(30) Foreign Application Priority Data
Jun. 15, 2021 (GB) 2108538.6

(51) Int. Cl. G16H 50/30; G16B 20/00

(52) U.S. Cl. CPC G16H 50/30; G16B 20/00

(57) ABSTRACT

The present invention relates to methods of determining an appropriate time interval for further diagnostic testing for prostate cancer in an individual having low total blood prostate specific antigen (PSA) levels.

### FIELD

The present invention relates to various methods of determining an appropriate time interval for further diagnostic testing for prostate cancer in an individual having low total blood prostate specific antigen (PSA) levels.

### BACKGROUND

According to the European Association of Urology (EAU), prostate cancer (PC) is the second most commonly diagnosed cancer in men, with an estimated 1.1 million diagnoses worldwide in 2012, accounting for 15% of all cancers diagnosed. It is therefore a major health concern in men, with incidence mainly dependent on age [1].

Predicting prostate cancer early can potentially reduce suffering and save lives, and calls for refined and individualised test results for subjects with low prostate specific antigen (PSA) levels have been made [2, 3].

Being falsely identified as having greater risk of prostate cancer in turn increases the patient's risk due to overdiagnosis or overtreatment, e.g. due to complications of having unnecessary biopsies performed [4, 5, 6]. The EAU states that there is strong evidence to “offer an individualised risk-adapted strategy for early detection to a well-informed man with a good performance status and a life-expectancy of at least ten to fifteen years” [2].

Clinical testing for prostate cancer has so far focused on the measurement of PSA [2]. Although this entity carries information that can be indicative of disease, it is by itself an uncertain basis for classification [2], as PSA is not solely elevated when prostate cancer is present.

Consequently, early detection strategies based on total PSA concentration alone remain a controversial topic. Stattin et al. studied the development of PC in males in Northern Sweden and concluded that “widespread PSA testing at age 40 cannot be justified” [7]. More recently, Pálsdóttir et al. studied occurrence of PC and recurrent testing for total PSA concentration in males in Stockholm, Sweden, and concluded that shorter intervals of testing can lead to lower risks of developing prostate cancer but also increase the risks of having negative biopsies performed [3].

### DESCRIPTION OF THE INVENTION

Therefore, the inventors have herein identified a need for an improved system of providing recommendations for the recurrence of diagnostic testing, i.e. the time to the next non-invasive test, rather than recommendations for biopsies. By doing so, the inventors aimed herein to facilitate the identification of those individuals that would benefit from conducting the next test earlier rather than later, while minimising the risk of unnecessary biopsies. The inventors have also determined herein a method of identifying individuals at low risk who would benefit from conducting the next test later.

Herein, the inventors present for the first time a method for determining an appropriate time interval for further diagnostic testing for prostate cancer in individuals with low PSA levels, by stratifying individuals in this group into three categories (normal risk, moderately low risk, and low risk) each with a different recommended time until a further diagnostic test is carried out.

The methods described herein facilitate early detection in individuals who currently are deemed to have a “low risk” for prostate cancer due to a low total PSA concentration, whist preventing any unnecessary investigations or treatments, by combining various input data based on PSA and genetic factors.

The present invention is based on the finding that the combination of data relating at least to total PSA and different single nucleotide polymorphisms (SNPs) specific to prostate cancer allows the stratification of risk of developing prostate cancer in individuals who have a low total PSA, thereby allowing more appropriate timing of further testing based on the risk of developing prostate cancer in the future.

This finding is advantageous as it results in less unnecessary testing (with fewer unnecessary treatments then being performed as a result of fewer false positive diagnoses). It also ensures that individuals at higher risk of developing prostate cancer in the future are tested more regularly, therefore ensuring prostate cancers are diagnosed as early as possible and are therefore easier to treat, leading to improved survival.

In a first aspect, the invention provides a method for determining an appropriate time interval for further diagnostic testing for prostate cancer in an individual having a total blood prostate-specific antigen (PSA) concentration (measured in unfractionated blood, serum or plasma, particularly serum or plasma) of less than or equal to 2 ng/ml, the method comprising the following steps:
a) providing at least one biological sample from the individual;
b) in said biological sample(s) analysing:
   a. the concentration of free PSA and total PSA; and/or
   b. a category of SNPs related to prostate cancer (SNPpc) by measuring the presence or absence of one or two risk allele(s) of each of a plurality of SNPpc of said category of SNPpc;
c) determining at least one of:
   c. the quotient of free PSA/total PSA; and
   d. a composite value based on a combination of the data regarding the SNPs related to prostate cancer, said composite value representing the risk of developing prostate cancer wherein the composite value is formed from at least 45 SNPs;
d) comparing the quotient of free PSA/total PSA to a pre-determined cut-off value established through analysis of cohort data from subjects with prostate cancer with known free PSA and total PSA; and/or comparing the SNP composite value to a pre-determined cut-off value established through analysis of cohort data from subjects with prostate cancer; and
e) classifying the individual as being at normal risk of prostate cancer according to one or more of the following criteria:
   i) the PSA quotient (free PSA (ng/ml)/total PSA (ng/ml)) is equal to or less than the pre-determined cut off value and the total PSA is from 1.3 to 1.5 ng/ml;
   ii) the SNP composite value is greater than the pre-determined cut off value and the total PSA value is greater than or equal to 1 ng/ml;
f) recommending that the individual should have a further prostate cancer diagnostic test within a first time period if one or both of the criteria in step (e) are satisfied.

In a second aspect, the invention provides a method for determining an appropriate time interval for further diagnostic testing for prostate cancer in an individual having a total blood prostate-specific antigen (PSA) concentration (measured in unfractionated blood, serum or plasma, particularly serum or plasma) of less than 1 ng/ml, the method comprising the following steps:
a) providing at least one biological sample from the individual;
b) in said biological sample(s) analysing:
   a. the concentration of total PSA; and/or
   b. a category of SNPs related to prostate cancer (SNPpc) by measuring the presence or absence of one or two risk allele(s) of each of a plurality of SNPpc of said category of SNPpc;
c) determining the age of the individual and/or the following:
   c. the total PSA concentration; and
   d. a composite value based on a combination of the data regarding the SNPs related to prostate cancer, said composite value representing the risk of developing prostate cancer, wherein the composite value is formed from at least 45 SNPs;
d) comparing the SNP composite value to a pre-determined cut-off value established through analysis of cohort data from subjects with prostate cancer; and
e) classifying the individual as being at low risk of prostate cancer according to one or more of the following criteria:
   i) the age of the individual is 80 years or greater;
   ii) the total PSA value is less than 1 ng/ml and the SNP composite value is lower than the predetermined cut-off value;
f) recommending that the individual should have a further prostate cancer diagnostic test within a second time period if one or both of the criteria in step (e) are satisfied.

In a third aspect, the invention provides a method for determining an appropriate time interval for further diagnostic testing for prostate cancer in an individual having a total blood prostate-specific antigen (PSA) concentration (measured in unfractionated blood, serum or plasma, particularly serum or plasma) of less than 1 ng/ml, the method comprising the following steps:
a) providing at least one biological sample from the individual;
b) in said biological sample(s) analysing:
   a. the concentration of total PSA; and/or
   b. a category of SNPs related to prostate cancer (SNPpc) by measuring the presence or absence of one or two risk allele(s) of each of a plurality of SNPpc of said category of SNPpc;
c) determining:
   c. the total PSA concentration; and
   d. a composite value based on a combination of the data regarding the SNPs related to prostate cancer, said composite value representing the risk of developing prostate cancer, wherein the composite value is formed from at least 45 SNPs;
d) comparing the SNP composite value to a pre-determined cut-off value established through analysis of cohort data from subjects with prostate cancer; and
e) classifying the individual as being at moderately low risk of prostate cancer according to the following criteria:
   i) the total PSA value is less than 1 ng/ml and the SNP composite value is higher than the predetermined cut-off value;
f) recommending that the individual should have a further prostate cancer diagnostic test within a third time period if the criteria in step (e) is satisfied.

By “prostate cancer” we include all types of cancer that affect the prostate, for example adenocarcinoma, squamous cell cancer, small cell cancers, neuroendocrine tumours, and sarcomas.

The term “PSA” refers to serum prostate specific antigen in general. PSA is also known as gamma-seminoprotein, kallikrein-3 (KLK3) or P-30 antigen. PSA is a glycoprotein enzyme encoded in humans by the KLK3 gene. It is secreted by the epithelial cells of the prostate gland and is found in the serum of healthy men in small quantities.

PSA is often elevated in the serum of men with prostate cancer, but is not a unique biomarker of prostate cancer, as it may indicate other issues such as prostatitis. PSA alone is therefore generally not considered a reliable indicator of the presence of prostate cancer.

PSA exists in different forms, where the term “free PSA” refers to PSA that is unbound or not bound to another molecule, the term “bound PSA” refers to PSA that is bound or complexed to another molecule, and finally the term “total PSA” refers to the sum of free PSA and bound (complexed) PSA. The term “F/T PSA” is the ratio of unbound PSA to total PSA.

There are also molecular derivatives of PSA, where the term “proPSA” refers to a precursor inactive form of PSA and “intact PSA” refers to an additional form of proPSA that is found intact and inactive.

The quantification of presence or concentration of biomarkers (e.g. PSA) in a biological sample can be made in many different ways. One common method is the use of enzyme linked immunosorbent assays (ELISA) which uses antibodies and a calibration curve to assess the presence and the concentration of a selected biomarker. ELISA assays are common and known in the art, as evident from the publication “Association between saliva PSA and serum PSA in conditions with prostate adenocarcinoma.” By Shiiki N and co-authors, published in Bio markers. 2011 September; 16(6):498-503, which is incorporated by reference herein.

Another common method is the use of a microarray assay for the quantification of presence or concentration of biomarkers in a biological sample. A typical microarray assay comprises a flat glass slide onto which a plurality of different capture reagents (typically an antibody) each selected to specifically capture one type of biomarker is attached in non-overlapping areas on one side of the slide. The biological sample is allowed to contact, for a defined period of time, the area where said capture reagents are located, followed by washing the area of capture reagents. At this point, in case the sought-after biomarker was present in the biological sample, the corresponding capture reagent will have captured a fraction of the sought-after biomarker and keep it attached to the glass slide also after the wash. Next, a set of detection reagents are added to the area of capture reagents (which now potentially holds biomarkers bound), said detection reagents being capable of (i) binding to the biomarker as presented on the glass slide and (ii) producing a detectable signal (normally through conjugation to a fluorescent dye). It is typically required that one detection reagent per biomarker is added to the glass slide.

There are many other methods capable of quantifying the presence or concentration of a biomarker, including, but not limited to, immunoprecipitation assays, immunofluorescence assays, radio-immuno-assays, and mass spectrometry using matrix-assisted laser desorption/ionization (MALDI), to mention a few examples.

In all aspects of the present invention, the individual has a total PSA concentration of 2 ng/ml or less. In some aspects of the invention, the individual has a total PSA concentration of 1.5 ng/ml or less. In some aspects of the invention, the individual has a total PSA concentration of 1 ng/ml or less.

By “total PSA” we mean the total PSA measured in blood (i.e. total blood PSA). Specifically, this includes the total PSA measured in unfractionated blood or serum or plasma, particularly serum or plasma. The skilled person would be aware of the common techniques for measurement of total PSA available in the art, and would be aware of how to measure total PSA in any of the blood sample types described herein.

In some embodiments, the individual has a total PSA concentration that is significantly lower than a standard general population PSA cut-off value for indicating a presence of prostate cancer.

“Significantly lower” in this regard may e.g. be at least about 10% lower than a standard cut-off PSA value, such as at least about 30%, 35%, 40%, 50%, 60% or even 70% lower than a standard cut-off value, such as at least about 30%, 35%, 40%, 50%, 60% or even 70% lower than e.g. about 4.0 ng/ml or 3.0 ng/ml depending on region.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

The PSA quotient is calculated using the following formula: free PSA/total PSA. The PSA quotient is a measure of how much of the total PSA is unbound or not bound to another molecule.

Methods of measuring the free PSA, bound PSA and total PSA are well known in the art. For example, medical and hospital laboratories will be able to measure total PSA and free PSA using standard instrumentation (for example instruments produced by Roche, Abbott, Siemens and ThermoFisher Scientific). Particular examples of machines that are routinely used to measure free PSA, bound PSA and total PSA include Abbott Architect, BioMerieux Vidas, BioMerieux VIDAS TPSA, Siemens Centaur XP/XPT/Classic, Siemens Immulite 2000/2500 1st generation, Siemens Immulite 1000 1st generation, Beckman DxI standardisd to Hybritech, Beckman DxI800 standardised to WHO IRP96/670, Roche Elecsys, Diasorin Liaison, Beckman Access standardised to Hybritech, Beckman Access standardised to WHO IRP96/670, Tosoh Series, Vitros ECi, CIS RIA Coated tube, Roche Cobas 4000/E411 and Roche Cobas e601/602, Monobind Inc. ELISA/CLIA, Ortho Vitros 3600/5600/ECi PSA II, Siemens Centaur CP, Diasorin Liaison XL, Siemens Atellica IM, Abbott Alinity I and Roche Cobas e801. All such machines are suitable for measuring PSA concentration (free, bound and/or total) in accordance with the methods of the invention. More particular instruments that may be mentioned include those manufactured by Roche (including Roche Cobas 4000/E411, Roche Cobas e601/602, Roche Elecsys and Roche Cobas e801), Siemens (including Siemens Centaur XP/XPT/Classic, Siemens Immulite 2000/2500 1st generation, Siemens Immulite 1000 1st generation, Siemens Centaur CP and Siemens Atellica IM), and Abbott (including Abbott Architect and Abbott Alinity I).

Accordingly, various platforms exist for measuring PSA, however despite being calibrated to World Health Organisation standards, these platforms may still produce discordant results depending on the platform used (see Boegemann et al. (2018), Discordant prostate specific antigen test results despite WHO assay standardisation, Intl J Biol Markers, 33(3): 285-282).

This notwithstanding, measurements obtained using the different platforms and methodologies are routinely treated as comparable by medical practitioners (e.g. in hospitals and primary care). Therefore, any technique and platform may be used to measure the concentration of PSA (free, bound and total) in accordance with the invention. For example, in accordance with the methods of the invention, PSA concentration (free, bound and total) may be determined in unfractionated blood, serum or plasma by many different methods, including but not limited to, chemiluminescence using magnetic particles (magnetic particle-based chemiluminescent immunoassay), Enzyme-Linked Immunosorbent Assay (ELISA) and Time-Resolved Amplified Cryptate Emission (TRACE). Of these techniques, chemiluminescence using magnetic particles is preferred.

In particular, in the methods of the invention, values for PSA concentration (free, total and bound) may be determined by chemiluminescence using magnetic particles (magnetic particle-based chemiluminescent immunoassay). Suitable instruments for this measurement include those manufactured by Roche (including Roche Cobas 4000/E411, Roche Cobas e601/602, Roche Elecsys and Roche Cobas e801), Siemens (including Siemens Centaur XP/XPT/Classic, Siemens Immulite 2000/2500 1st generation, Siemens Immulite 1000 1st generation, Siemens Centaur CP and Siemens Atellica IM), and Abbott (including Abbott Architect and Abbott Alinity I). Such measurements may be taken in unfractionated blood, serum or plasma (preferably serum or plasma).

In a further particular embodiment, the PSA concentration may be measured by TRACE, for example using a Thermo Fisher KRYPTOR® analyser. More particularly, the PSA concentration may be measured by TRACE, for example using a Thermo Fisher KRYPTOR® analyser, in a serum or plasma sample (e.g plasma).

The term “diagnostic testing” refers to the testing and detection of the presence or nature of a pathologic condition, in this instance, prostate cancer. It may be used interchangeably with “diagnostic method”. Diagnostic methods differ in their sensitivity and specificity.

One measure of the usefulness of a diagnostic method is “area under the receiver-operator characteristic curve”, which is commonly known as ROC-AUC statistics. This widely accepted measure takes into account both the sensitivity and specificity of the tool. The ROC-AUC measure typically ranges from 0.5 to 1.0, where a value of 0.5 indicates the tool has no diagnostic value and a value of 1.0 indicates the tool has 100% sensitivity and 100% specificity.

The term “sensitivity” refers to the proportion of all subjects with PCa that are correctly identified as such (which is equal to the number of true positives divided by the sum of the number of true positives and false negatives).

The term “specificity” refers to the proportion of all subjects healthy with respect to PCa (i.e. not having PCa) that are correctly identified as such (which is equal to the number of true negatives divided by the sum of the number of true negatives and false positives).

The term “biological sample” as used herein refers to any type of sample that can be obtained from an individual for diagnostic testing. They include but are not limited to: unfractionated blood, plasma, serum, saliva and urine. In some preferred embodiments, the biological sample is unfractionated blood or serum or plasma. In some embodiments, the biological sample is serum or plasma. A skilled person will understand that a serum or plasma sample is derived from an unfractionated blood sample obtained from the individual and processed using standard techniques to obtain serum or plasma.

In the methods of the present invention, the different parameters (e.g. PSA, presence of SNPs) may be measured from the same biological sample or different biological samples taken from the same individual. These samples may be taken at the same time or at different times.

In some preferred embodiments of the methods of the present invention, when free and/or total PSA is measured, the biological sample is a serum sample or a plasma sample. In these preferred embodiments, the presence or absence of SNPs may be determined using an unfractionated blood sample (for instance by utilising harvested human cells, for example remaining blood cells after having fractionated the blood) or a saliva sample. It will also be understood by the skilled person that SNPs can be detected from any biological sample containing the DNA of the individual.

In some preferred embodiments, the methods of the present invention utilise PSA measurements made from serum or plasma samples and measurement of SNPs made from another sample type (e.g. unfractionated blood or saliva).

In some embodiments, an unfractionated blood sample is obtained from the individual and is: (i) used to obtain a serum sample or plasma sample for measurement of PSA from a serum or plasma sample; and (ii) used to determine the presence or absence of SNPs directly from the unfractionated blood sample.

By “individual” we include any subject that can or may be diagnosed with prostate cancer. In preferred embodiments, the individual is a human. In preferred embodiments, the individual is a male. In preferred embodiments, the individual has a prostate.

In some embodiments, the individual is over 45 years of age, for example the individual is over 50 years of age; is over 55 years of age; is over 60 years of age; is over 65 years of age; is over 70 years of age; is over 75 years of age; or is about 80 years of age. Preferably, the individual is 80 years of age or younger.

In all aspects of the invention, the individual has a low level of blood total PSA (which may be measured in unfractionated blood, serum or plasma, particularly serum or plasma). By “low level” we mean the individual has a level of less than or equal to 2 ng/ml total PSA as measured by a standard PSA test. In some embodiments, “low level” means that an individual has a total PSA level of 1.5 ng/ml or less as measured by a standard PSA test. By “standard PSA test” we mean a test that measures the total PSA concentration in serum or plasma.

It is important to determine when such individuals with low PSA levels should have a further diagnostic test based on their risk of developing prostate cancer. To test these individuals too frequently risks unnecessary testing and biopsies being performed, which can lead to unnecessary diagnosis and treatment. To test these individuals too infrequently risks missing diagnoses that could have been made earlier, and are thus easier to treat.

The term “single nucleotide polymorphism” (SNP) refers to the genetic properties of a defined locus in the genetic code of an individual. A SNP can be related to increased risk of prostate cancer, and can hence be used for diagnostic assessment of an individual, or in the case of the present invention, to determine the risk of developing prostate cancer in the future, thereby allowing safer prediction of when the next diagnostic test should be.

The Single Nucleotide Polymorphism Database (dbSNP) is an archive for genetic variation within and across different species developed and hosted by the National Center for Biotechnology Information (NCBI) in collaboration with the National Human Genome Research Institute (NHGRI), both located in the US. Although the name of the database implies a collection of one class of polymorphisms only (i.e., single nucleotide polymorphisms (SNP)), it in fact contains a range of molecular variation. Every unique submitted SNP record receives a reference SNP ID number (“rs #”; “refSNP cluster”). In this application, SNP are mainly identified using rs# numbers.

Accordingly, within the present application, SNP is used to refer to the range of molecular variation as included in the dbSNP, rather than only single nucleotide polymorphisms. For the purpose of the present application, the terms “SNP” and “SNPs” may be used interchangeably, and may be used to describe the singular and/or the plural of “single nucleotide polymorphism”.

The term “composite value” refers to the combination of data related to a parameter category into a representative value for said parameter category. The combination of data can typically be performed according to one or more predetermined equations. A composite value is the output of the combination of data according to one or more predetermined equations. The different equations are applicable for different measurement results (i.e. data), depending on for which subsets of the members of the parameter category that data are available.

In the context of the present application, a composite value is calculated based on the combination of data relating to prostate cancer related SNPs. The composite value can be defined as an accumulation of risks associated with each contributing SNP.

One non-limiting example of a method to form a composite value for a particular parameter category is to use the average of the available results for the members of said category. The term “composite value” is sometimes referred to as “score” in the present application, and as used herein refers to a “genetic composite value” (or “genetic score”), and more specifically an “SNP composite value”. In some embodiments, the composite value of the genetic score is assigned a pre-determined cut-off value, above or below which an individual may be assigned a particular category (subject to other criteria being met).

In the present invention, the pre-determined cut-off value for the SNP composite value is calculated as the 90^{th} percentile of the genetic score in the original group of patients used to define the method. The original group of patients comprised a large number of individuals with PSA >1.5. The pre-determined cut-off value does therefore not reflect the 90^{th} percentile of a cohort of individuals with PSA <2. In some embodiments, the pre-determined cut off value of the SNP composite value is determined as the 90^{th} percentile of the SNP composite value of the original cohort of patients used to determine the cut-off value. In addition, the pre-determined cut-off value for the SNP composite value may differ slightly depending on the set and number of SNPs used to construct the genetic score.

In some embodiments, this pre-determined cut off value of the SNP composite value is set to 1.0 based on the genetic score used in the original cohort (based on a genetic score from 101 SNPs) which corresponds to 0.784 for genetic score calculated on the 45-50 SNP in some embodiments of this invention. Therefore, in some alternative embodiments, the pre-determined cut off value of the SNP composite value is set to 0.784.

The quantification of the presence of SNPs through analysis of a biological sample may be achieved by a variety of different methods known in the art.

For example, the quantification of presence of SNPs through the analysis of a biological sample (for example, an unfractionated blood sample or saliva sample as discussed herein) may involve MALDI mass spectrometry analysis based on allele-specific primer extensions. Therefore, the measurement of the presence or absence of SNPs may be conducted by MALDI mass spectrometry in some embodiments.

In some other embodiments, the measurement of the presence or absence of SNPs may be conducted by a PCR-based SNP genotyping assay, for example utilising Taq polymerase. An example of such a method is TaqMan® SNP genotyping, which utilises the 5′ nuclease activity of Taq polymerase to generate a fluorescent signal from a dye-quencher labelled primer during PCR that is indicative of the presence of a particular SNP that hybridises to the primer designed to hybridise to a target sequence.

Although the combining of data can be performed in different ways, a typical procedure according to the present invention can be illustrated in the following non-limiting manner.

The genetic score (i.e. the genetic composite value, or more specifically the SNP composite value) calculation is typically based on a predetermined odds ratio for each individual SNP included in a parameter category. For each SNP the odds ratio, i.e. the likelihood that an individual who carries a SNP (i.e. has the risk allele defined by the SNP) has the disease or condition under study, is determined in advance. Determination of the odds ratio for a SNP is usually done in large prospective studies involving thousands of subjects with known conditions or diseases. The skilled person would readily be able to calculate an odds ratio using techniques known in the art, see for example, the Odds Ratio Wikipedia page.

The genetic score for an individual can, as a non-limiting example, be computed according to the following algorithm by processing each SNP in the following manner. For each SNP the individual may carry two SNP risk alleles (homozygous positive for said SNP), or one risk allele (heterozygous positive for said SNP) or zero risk alleles (homozygous negative for said SNP). In case an SNP is located on a chromosome available only in one copy (such as the Y chromosome), the SNP can only carry one allele (either a risk allele or a normal/expected allele) which may be referred to as hemizygous. Hemizygous SNPs may need scaling in subsequent calculations because the number of risk alleles can only be 0 or 1.

The number of alleles for a SNP is multiplied with the natural logarithm of the odds ratio for said SNP to form a risk assessment value for that particular SNP. This means that an individual who is negative for a particular SNP (i.e. has zero SNP risk alleles) will have no risk contribution from said particular SNP. This procedure is repeated for all SNP for which measurement data is available. When all risk assessment values have been calculated, the average of the risk contribution for the SNP for which measurement data are available is calculated and is used as the genetic score for said individual, i.e. the genetics composite value with respect to a certain category of SNPs. This procedure may clearly be applied regardless of how many SNP members belong to the SNP category. This procedure may further be applied to a small subset of defined (often very high-risk or very low-risk) SNPs to define if an individual is member of a particular high-risk or low-risk subgroup within each of the groups defined herein.

Suitable SNPs related to prostate cancer that are preferably utilised in the methods of the present invention include:
rs138213197; rs7818556; rs6983267; rs10993994; rs12793759; rs16901979; rs9911515; rs1016343; rs7106762; rs6579002; rs16860513; rs5945619; rs16902094; rs10896437; rs651164; rs7679673; rs13265330; rs2047408; rs10107982; rs620861; rs9297746; rs1992833; rs7213769; rs2710647; rs888507; rs17021918; rs12500426; rs2028900; rs7102758; rs16901922; rs6062509; rs2659051; rs12543663; rs4699312; rs11091768; rs3120137; rs6794467; rs10086908; rs2315654; rs12151618; rs747745; rs1009; rs2132276; rs2735839; rs11568818; rs684232; rs9364554; rs2660753; rs10807843; rs1933488; rs17467139; rs12947919; rs2331780; rs1894292; rs2107131; rs6545962; rs11649743; rs758643; rs2297434; rs902774; rs17224342; rs5918762; rs17138478; rs3019779; rs1873555; rs12946864; rs12475433; rs3765065; rs4871779; rs10875943; rs11601037; rs6489721; rs11168936; rs9297756; rs11900952; rs6569371; rs7752029; rs5934705; rs3745233; rs1482679; rs749264; rs6625760; rs5978944; rs2366711; rs5935063; rs10199796; rs2473057; rs4925094 rs3096702; rs12490248; rs4245739; rs10094059; rs306801; rs2823118; rs2025645; rs9359428; rs10178804; rs6090461; rs2270785; rs16901841; rs2465796.

In some preferred embodiments of the aspects disclosed herein, the method comprises forming the composite value based on at least 45 SNPs selected from the following list:
rs138213197; rs7818556; rs6983267; rs10993994; rs12793759; rs16901979; rs9911515; rs1016343; rs7106762; rs6579002; rs16860513; rs5945619; rs16902094; rs10896437; rs651164; rs7679673; rs13265330; rs2047408; rs10107982; rs620861; rs9297746; rs1992833; rs7213769; rs2710647; rs888507; rs17021918; rs12500426; rs2028900; rs7102758; rs16901922; rs6062509; rs2659051; rs12543663; rs4699312; rs11091768; rs3120137; rs6794467; rs10086908; rs2315654; rs12151618; rs747745; rs1009; rs2132276; rs2735839; rs11568818; rs684232; rs9364554; rs2660753; rs10807843; rs1933488.

In some particularly preferred embodiments, the method of each aspect involves measuring all 50 of the SNPs listed above.

In some additional embodiments, the method may further involve including one or more SNPs from the following list:
rs17467139; rs12947919; rs2331780; rs1894292; rs2107131; rs6545962; rs11649743; rs758643; rs2297434; rs902774; rs17224342; rs5918762; rs17138478; rs3019779; rs1873555; rs12946864; rs12475433; rs3765065; rs4871779; rs10875943; rs11601037; rs6489721; rs11168936; rs9297756; rs11900952; rs6569371; rs7752029; rs5934705; rs3745233; rs1482679; rs749264; rs6625760; rs5978944; rs2366711; rs5935063; rs10199796; rs2473057; rs4925094; rs3096702; rs12490248; rs4245739; rs10094059; rs306801; rs2823118; rs2025645; rs9359428; rs10178804; rs6090461; rs2270785; rs16901841; rs2465796.

In some embodiments, the method involves measuring all 101 SNPs from the following list:
rs138213197; rs7818556; rs6983267; rs10993994; rs12793759; rs16901979; rs9911515; rs1016343; rs7106762; rs6579002; rs16860513; rs5945619; rs16902094; rs10896437; rs651164; rs7679673; rs13265330; rs2047408; rs10107982; rs620861; rs9297746; rs1992833; rs7213769; rs2710647; rs888507; rs17021918; rs12500426; rs2028900; rs7102758; rs16901922; rs6062509; rs2659051; rs12543663; rs4699312; rs11091768; rs3120137; rs6794467; rs10086908; rs2315654; rs12151618; rs747745; rs1009; rs2132276; rs2735839; rs11568818; rs684232; rs9364554; rs2660753; rs10807843; rs1933488; rs17467139; rs12947919; rs2331780; rs1894292; rs2107131; rs6545962; rs11649743; rs758643; rs2297434; rs902774; rs17224342; rs5918762; rs17138478; rs3019779; rs1873555; rs12946864; rs12475433; rs3765065; rs4871779; rs10875943; rs11601037; rs6489721; rs11168936; rs9297756; rs11900952; rs6569371; rs7752029; rs5934705; rs3745233; rs1482679; rs749264; rs6625760; rs5978944; rs2366711; rs5935063; rs10199796; rs2473057; rs4925094 rs3096702; rs12490248; rs4245739; rs10094059; rs306801; rs2823118; rs2025645; rs9359428; rs10178804; rs6090461; rs2270785; rs16901841; rs2465796.

Genetic risk scores (i.e. genetic scores, or genetic composite values, more particularly SNP composite values) are also insensitive to small losses of data due to for example unforeseen technical problems, human error, or any other unexpected and uncommon reason. The contribution of one SNP to the risk score is typically not correlated to any other SNP. In the case of SNP, the risk change due to each SNP is small, but by using multiple SNP related to a condition in concert, the risk change for said condition becomes large enough for having an impact on the model performance.

The preferred number of SNPs to form a composite value herein is at least 45 SNPs. This means that the impact of any single SNP on the total result is typically small, and the omission of a few SNP will typically not alter the overall genetic score risk assessment in any large manner, i.e. will typically not alter the SNP composite value to a significant extent. In current state of the art, the typical data loss in the large-scale genetic measurements is on the order of 1-2%, meaning that if a genetic score is composed of 100 different SNP, the typical genetic characterization of an individual would provide information about 98-99 of these SNPs. The present model as such, as discovered in the work of the present invention, can however withstand a larger loss or lack of data, such as 5% loss of information. In this sense, the combination of data regarding SNPpc is at least partially redundant.

Consequently, also with respect to genetic markers (SNPs), the present invention relates to a method that is based on a redundantly designed combination of data. The method allows disregarding at least about 5% of the SNPpc when forming the SNP composite value. In other words, the method allows that said SNPpc composite value is formed from data regarding less than all SNPpc of the SNPpc category, more specifically data regarding a subset of at most 95% of said SNPpc. As the skilled person will appreciate, this will be equivalent to a method where data regarding a subset of at most 95% of said SNPpc are required to form said SNPpc composite value.

The methods of the present invention involve comparing various measured or calculated values to pre-determined cut-off values. The choice of cut-off value depends on many factors, including but not limited to the risk of the disease developing in an individual as such and the risk associated with testing an individual at low risk of prostate cancer too frequently (increasing the risk of false positives). In the general case, a predictive model is usually a monotonic function Y=f(x1, x2, . . . , xN) where the estimated risk of having the disease is correlated with the increasing value of Y.

This means that if the cut-off value is set at a low level, the test may lead to a larger number of false positive results (i.e. due to increased testing in lower risk individuals), but will on the other hand detect most individuals that have a higher risk of developing the disease. If the cut-off level is set at a high value the opposite occurs where individuals having a Y value above the cut-off level will with very high probability of developing the disease, but a large number of individuals that may be at risk of developing the disease will be categorised as low risk. The choice of cut-off level depends on many factors, including the socio-economic outcome of balancing (a) missing individuals with the disease and (b) treating individuals without the disease.

By utilising the methods of the first, second and third aspects, the present invention allows the classification of individuals who have a total PSA of 2 ng/ml or less into three groups:
1. Normal risk for prostate cancer;
2. Low risk for prostate cancer;
3. Moderately low risk for prostate cancer.

This classification works based on determining whether the various parameters of the first, second and third aspects are met. Based on this classification, it is possible to stratify individuals who have low PSA values into groups based on risk of developing prostate cancer, and therefore recommend that the next test should be performed within a time period that is appropriate to that level of risk, such that the risk of missing a diagnosis of prostate cancer is minimised.

The advantage of this over previous methods that have recommended reducing PSA testing intervals in individuals with low PSA values is that it also incorporates other risk factors, such as genetic risk factors (represented by the SNP based composite value), providing a greater level of detail than simply measuring PSA alone. This minimises the risk of either testing a low-risk individual too frequently or missing a diagnosis of prostate cancer by testing a higher-risk individual too infrequently.

By “further prostate cancer diagnostic test” we include any diagnostic test capable of diagnosing prostate cancer within the defined time period. Prostate cancer diagnostic tests are known in the art.

In preferred embodiments, this further diagnostic test is an “advanced” diagnostic test. In some preferred embodiments the advanced diagnostic test has a false positive rate of less than about 35-40%. In some embodiments, the diagnostic test has a false positive rate of about 36% or less. The skilled person will readily be able to determine the false positive rate of a diagnostic test using methods well known in the art, as explained in Läkartidningen. 2018,115: FCDT, Men who want to get tested for prostate cancer—a structured model (https://lakartidningen.se/klinik-och-vetenskap-1/artiklar-1/vardutveckling/2018/10/man-som-vill-testa-sig-for-prostatacancer-en-strukturerad-modell/#).

In some additional or alternative embodiments, the advanced prostate cancer test is selected from the following list of non-limiting examples: Stockholm3; 4K; Prostate Health Index (PHI); or ExoDx Prostate IntelliScore (EPI). Examples of these tests are discussed further in the European Association of Urology Prostate Cancer Guidelines (Section 5.2.3) (https://uroweb.org/guideline/prostate-cancer/#5) or the proposed US MCD policy (https://www.cms.gov/medicare-coverage-database/details/lcd-details.aspx?LCDId=38853).

The use of such advanced tests in the methods of the invention has the advantage of further reducing the risk of overdiagnosis and unnecessary invasive treatments and testing.

In some embodiments, the individual may have more than one advanced prostate cancer diagnostic test within the time period specified in step (f) of each aspect.

In some embodiments, the individual may have further prostate cancer diagnostic tests outside of the time period specified in step (f) of each aspect.

In some embodiments, the individual may also have prostate cancer diagnostic tests that are not advanced prostate cancer diagnostic tests within the time period specified in step (f) of each aspect.

Examples of prostate cancer diagnostic tests that are not advanced tests include but are not limited to: PSA blood tests and digital rectal examination.

These multiple tests may be of the same type (i.e. a repeat test) or different types from the list above. These multiple tests may also be carried out at different times within the range of time periods specified in step (f) of each aspect.

By “first time period” we mean that an individual falling into the “normal risk” group as defined according to the first aspect of the invention will be recommended to have a further diagnostic test within a time period from about 2 years to about 4 years. In a preferred embodiment, the further diagnostic test is carried out within a time period from 2 years to 4 years.

By “second time period” we mean that an individual falling into the “low risk” group as defined according to the first or second aspect of the invention will be recommended to have a further diagnostic test within a time period from about 6 years to about 10 years. In a preferred embodiment, the further diagnostic test is carried out within a time period from 6 years to 10 years.

By “third time period” we mean that an individual falling into the “moderately low risk” group as defined according to the first, second, or third aspects of the invention will be recommended to have a further diagnostic test within a time period from about 4 years to about 6 years. In a preferred embodiment, the further diagnostic test is carried out within a time period from 4 years to 6 years.

Within the time periods defined herein there is intended to be some flexibility in how the exact timing of the further test is determined. The exact timing of the test within the defined window can be determined by a skilled practitioner. The timing of further testing as defined herein is based on the observation that the volume of a prostate tumour doubles roughly every 2 years, so in individuals with low PSA it is reasonable to carry out a further test within certain multiples of the typical doubling time, with the time window determined using the risk-based approach defined herein. Prostate cancer is known to be a disease with slow progression, as discussed in the report “Observations on the doubling time of prostate cancer. The use of serial prostate-specific antigen in patients with untreated disease as a measure of increasing cancer volume”, by Schmid and co-authors as published in Cancer, Mar. 15, 1993; 71(6):2031-40, which is incorporated by reference herein. According to this report, the time for doubling of the tumour volume exceeds 24 months in a majority of the prostate cancers studied. Another report related to tumour volume in prostate cancer discloses that a 2-year doubling time for prostate cancer can be considered aggressive, while 3- and 4-year doubling times represent the typical growth rate of most diagnosed prostate cancers (“Impact of Life Expectancy and Tumor Doubling Time on the Clinical Significance of Prostate Cancer in Japan” by Egawa and co-authors, Jpn. J. Clin. Oncol. (1997) 27 (6): 394-40; incorporated by reference herein).

By “within a time period” we mean that the time period within which the further test should be carried out is measured from the time at which the initial PSA test (that measured a low PSA level of below 2 ng/ml) was carried out.

In some embodiments, the individual is diagnosed with prostate cancer following the further prostate cancer diagnostic test recommended in step (f). In some embodiments, further diagnostic testing (such as a biopsy) may be required to confirm said diagnosis.

In some embodiments, following a diagnosis of prostate cancer according to any of the methods described herein, the individual is treated for prostate cancer. Therefore, in some embodiments, the methods of the present invention comprise a further step of treating the individual for prostate cancer.

Treatments for prostate cancer are known in the art and can include one or more of the following: chemotherapy; surgery (i.e. prostatectomy); radiotherapy (external radiotherapy and brachytherapy); immunotherapy; hormone therapy; cryotherapy; thermotherapy; and targeted therapies. The skilled person will be aware of how to select an appropriate prostate cancer treatment based on the stage of prostate cancer at diagnosis, and other factors such as the age of the patient and any co-morbidities.

In some other embodiments, the individual is not diagnosed with prostate cancer following the further diagnostic test recommended in step (f). In these embodiments, the individual may be categorised again according to the methods of the present invention, and therefore be recommended a further diagnostic test within one of the time periods of step (f) of the first, second or third aspects of the invention.

Therefore, in some embodiments, the methods of the present invention can be repeated on the same individual, for example they may be repeated twice, three, four or five times.

In some other embodiments, the individual may not be categorised further according to the present invention (e.g. if their total PSA rises to above 2 ng/ml), and even if they are not diagnosed with prostate cancer, they may be recommended to have further diagnostic testing within a shorter time period, or may be recommended to have a prostate biopsy or an advanced prostate cancer diagnostic test.

As discussed above, the first aspect of the invention is a method for determining an appropriate time interval for further diagnostic testing for prostate cancer in an individual having a total blood prostate-specific antigen (PSA) concentration (measured in unfractionated blood, serum or plasma, particularly serum or plasma) of less than or equal to 2 ng/ml, the method comprising the following steps:
a) providing at least one biological sample from the individual;
b) in said biological sample(s) analysing:
   a. the concentration of free PSA and total PSA; and/or
   b. a category of SNPs related to prostate cancer (SNPpc) by measuring the presence or absence of one or two risk allele(s) of each of a plurality of SNPpc of said category of SNPpc;
c) determining at least one of:
   c. the quotient of free PSA/total PSA; and
   d. a composite value based on a combination of the data regarding the SNPs related to prostate cancer, said composite value representing the risk of developing prostate cancer wherein the composite value is formed from at least 45 SNPs;
d) comparing the quotient of free PSA/total PSA to a pre-determined cut-off value established through analysis of cohort data from subjects with prostate cancer with known free PSA and total PSA; and/or comparing the SNP composite value to a pre-determined cut-off value established through analysis of cohort data from subjects with prostate cancer; and
e) classifying the individual as being at normal risk of prostate cancer according to one or more of the following criteria:
   i. the PSA quotient (free PSA (ng/ml)/total PSA (ng/ml)) is equal to or less than the pre-determined cut off value and the total PSA is from 1.3 to 1.5 ng/ml;
   ii. the SNP composite value is greater than the pre-determined cut off value and the total PSA value is greater than or equal to 1 ng/ml;
f) recommending that the individual should have a further prostate cancer diagnostic test within a first time period if one or both of the criteria in step (e) are satisfied.

In some embodiments of the first aspect of the invention, the quotient of free PSA/total PSA is determined in addition to the total PSA concentration. As discussed herein, the PSA quotient is calculated by dividing the free PSA by the total PSA. In this embodiment, the PSA quotient is compared to a pre-determined cut-off value in order to determine whether the individual is at normal risk of prostate cancer.

In some embodiments, the pre-determined cut-off value of the PSA quotient is from about 0.10 to 0.12. In some embodiments, the pre-determined cut-off value of the PSA quotient is from about 0.105 to 0.115.

In some preferred embodiments, the pre-determined cut-off value of the PSA quotient is about 0.11.

For an individual to be categorised as being at normal risk of prostate cancer based on the PSA quotient as described above, the total PSA concentration must also be measured within a certain range. In this embodiment, the total PSA concentration must be from about 1.3 ng/ml to about 1.5 ng/ml.

The purpose of this combination of the PSA quotient and the total PSA is to account for uncertainty in free PSA measurements at a low total PSA (approaching 1 ng/ml), such that measurements of free PSA when the total PSA is lower than about 1.3 ng/ml are uncertain due to the sensitivity limits of the assay. In addition, different platforms for measuring free and total PSA have different systematic deviations (as discussed above), and therefore the range of 1.3 to 1.5 ng/ml is used in order to manage the effects of known systematic differences between different lab settings and equipment.

In some other embodiments when the individual is to be categorised as being at normal risk of prostate cancer based on the PSA quotient as described above, the total PSA concentration must also be measured within a range of from about 1.2 ng/ml to about 1.5 ng/ml. In some other embodiments, the total PSA concentration must also be measured within a range of from about 1.1 ng/ml to about 1.5 ng/ml. In some embodiments the total PSA concentration must also be measured within a range of from about 1.3 ng/ml to about 1.4 ng/ml. In some embodiments the total PSA concentration must also be measured within a range of from about 1.3 ng/ml to about 1.45 ng/ml. In some embodiments the total PSA concentration must also be measured within a range of from about 1.2 ng/ml to about 1.4 ng/ml. In some embodiments the total PSA concentration must also be measured within a range of from about 1.2 ng/ml to about 1.45 ng/ml. In some embodiments the total PSA concentration must also be measured within a range of from about 1.1 ng/ml to about 1.4 ng/ml. In some embodiments the total PSA concentration must also be measured within a range of from about 1.1 ng/ml to about 1.45 ng/ml.

In some additional or alternative embodiments, the individual is categorised as having a normal risk of prostate cancer if the composite value based on measuring the presence and absence of various SNPs is greater than or equal to a pre-determined cut off value.

This pre-determined cut off value is calculated based on comparing various sets of prostate cancer associated SNPs in data sets of individuals having diagnosed prostate cancer, to establish a set of SNPs representing a risk of prostate cancer. The SNP based composite value is calculated as described herein.

In some embodiments of the first aspect, the individual is categorised as being at normal risk of prostate cancer if the SNP based composite value is greater than or equal to the 90th percentile of the SNP composite value calculated from the in the original group of patients used to define the method (as discussed above). In some embodiments, the individual is categorised as being at normal risk of prostate cancer if the SNP based composite value is greater than or equal to 1.0 when calculated as described herein. In some other embodiments, the individual is categorised as being at normal risk of prostate cancer if the SNP based composite value is greater than or equal to 0.784 when calculated as described herein.

When the SNP composite value is greater than or equal to the pre-determined cut-off value, an individual is categorised as having a normal risk of prostate cancer if they also have a total PSA of greater than or equal to 1 ng/ml.

Therefore, in some embodiments, for an individual to be categorised as being at normal risk of prostate cancer, the individual must have a total PSA concentration of less than or equal to 2 ng/ml and:
Have a PSA quotient of less than or equal to 0.11 with a total PSA concentration of from 1.3 ng/ml to 1.5 ng/ml; and/or
Have an SNP based composite value of greater than or equal to 1 (or 0.784) with a total PSA concentration of greater than or equal to 1 ng/ml (i.e. from 1 ng/ml to 1.3 ng/ml).

If one or both of the conditions above are fulfilled, the individual falls into the “normal” risk category, and a further test according to step (f) is recommended within a first time period as defined herein.

In some additional embodiments, the first aspect of the invention also involves assessing the risk of the individual using the Prostate Cancer Prevention Trial (PCPT) equation. By “PCPT equation” we refer to a predictive model of prostate cancer developed during a large-scale prostate cancer diagnosis study conducted in the United States.

The PCPT model is described further in Thompson et al., Assessing prostate cancer risk: results from the Prostate Cancer Prevention Trial, J Natl Cancer Inst., 2006, 98(8): 529-34 (incorporated herein by reference). The PCPT model output may be expressed as risk of prostate cancer or risk of high-grade prostate cancer, for example.

The PCPT integrates several prostate cancer risk factors including: age; total PSA concentration; whether there is a family history of prostate cancer; and whether the individual has previously had a negative prostate cancer biopsy.

In some additional embodiments, the PCPT as used herein also integrates the race of the individual. The race may be categorised as white, African-American or other, although the race of the individual may not have a significant impact on the outcome of the PCPT equation.

Therefore, in some embodiments when the PCPT score is included in the method of the first aspect:
i) step b) further comprises assessing the individual's risk of having prostate cancer using the Prostate Cancer Prevention Trial (PCPT) equation;
ii) step d) further comprises comparing the risk of the individual having prostate cancer calculated using the PCPT equation to a predetermined reference value, representing the risk of developing prostate cancer;
iii) step e) further comprises classifying the individual as normal risk of prostate cancer if the PCPT equation score is above or equal to the reference value.

In some embodiments, the PCPT score is expressed as a percentage risk. The percentage risk, in some preferred embodiments, refers to a percentage risk of the individual having or developing high-grade prostate cancer disease (i.e. with a Gleason score of 7 or above).

By “Gleason score” we refer to the common grading system used to determine the aggressiveness of prostate cancer (see, for example, Wikipedia page on Gleason grading system). The Gleason score is usually determined by grading cells in a prostate biopsy from 1 (normal tissue) to 5 (high grade). One grade is assigned based on the most predominant grade of cells present, and a second grade is assigned to the second most predominant grade of cells present. These two grades are added together to reach a Gleason score of between 2 and 10. Typically, a Gleason score of 6 represents low grade cancer, 7 is intermediate grade and 8-10 is high grade cancer. A Gleason score of 7 and above is typically described as clinically significant prostate cancer, which corresponds to a score of 2 or greater in ISUP grading (International Society of Urological Pathology).

In some embodiments, the pre-determined reference value of the PCPT equation score referred to above is about 3% risk of having or developing prostate cancer. Therefore, in some embodiments, an individual categorised as having a normal risk of prostate cancer has a risk of having or developing prostate cancer with a Gleason score of 7 or above of greater than or equal to 3%. For example, in some embodiments, the percentage risk of having or developing prostate cancer with a Gleason score of 7 or above may be: 3% or above; 4% or above; 5% or above; 6% or above; 7% or above; 8% or above; 9% or above; or 10% or above. In some embodiments, the percentage risk of having or developing prostate cancer with a Gleason score of 7 or above may be from 3% to 11%.

In some other embodiments, the PCPT score may be expressed as a percentage risk of developing high risk prostate cancer.

In some embodiments, the individual does not meet any of the requirements of step (e) of the first aspect of the invention, and therefore is not categorised as having a normal risk of prostate cancer.

In this case, the individual is either classified as having a low risk of prostate cancer (and a further test is recommended within a second time period) or the individual is categorised as having a moderately low risk of prostate cancer (and a further test is recommended within a third time period).

Therefore, in some embodiments, step c) of the first aspect of the invention further comprises determining the age of the individual, and step e) further comprises classifying the individual as being of low risk of prostate cancer if one or more of the following applies:
i) the age of the individual is 80 years or greater;
ii) the total PSA value is less than 1 ng/ml and the SNP composite value is lower than the predetermined cut-off value;

and step f) further comprises recommending that such individuals should have a further prostate cancer diagnostic test within a second time period.

Therefore, if an individual is 80 years old or greater and has a total PSA value of 2 ng/ml or less, they are categorised as having a low risk of prostate cancer, regardless of their total PSA value and SNP composite value. In some additional embodiments, if an individual has a current life expectancy of 15 years or less and has a total PSA value of 2 ng/ml or less, they are categorised as having a low risk of prostate cancer, regardless of their total PSA value and SNP composite value.

In some other embodiments, the individual does not satisfy any of the criteria of step (e) described above. In this case, the method further comprises classifying the individual as being of moderately low risk of prostate cancer if the individual does not satisfy any of the criteria of step (e), and recommending that such individuals should have a further prostate cancer diagnostic test within a third time period as defined herein.

In some embodiments of the first aspect, the total PSA concentration is less than 2 ng/ml, for example the total PSA concentration may be: 1.9 ng/ml or less; 1.8 ng/ml or less; 1.7 ng/ml or less; 1.6 ng/ml or less; or about 1.5 ng/ml or less. In some preferred embodiments, the total PSA concentration is less than 1.5 ng/ml.

In these embodiments where the total PSA concentration of individuals in the method of the first aspect are 2 ng/ml or less, individuals with a total PSA concentration of greater than 1.5 ng/ml may be recommended to have an advanced prostate cancer diagnostic test immediately.

In some other embodiments, when the individual has a total PSA of greater than 1 ng/ml but less than 1.3 ng/ml, they will be categorised as normal risk or moderately low risk of prostate cancer. If their genetic score is less than or equal to the pre-determined cut-off value of the SNP composite value, the individual will be categorised as moderately low risk as defined herein. If their genetic score is greater than the pre-determined cut-off value of the SNP composite value, the individual will be categorised as normal risk as defined herein.

As discussed above, a second aspect of the invention is a method for determining an appropriate time interval for further diagnostic testing for prostate cancer in an individual having a total blood prostate-specific antigen (PSA) concentration (measured in unfractionated blood, serum or plasma, particularly serum or plasma) of less than 1 ng/ml, the method comprising the following steps:
a) providing at least one biological sample from the individual;
b) in said biological sample(s) analysing:
   a. the concentration of total PSA; and/or
   b. a category of SNPs related to prostate cancer (SNPpc) by measuring the presence or absence of one or two risk allele(s) of each of a plurality of SNPpc of said category of SNPpc;
c) determining the age of the individual and/or the following:
   c. the total PSA concentration; and
   d. a composite value based on a combination of the data regarding the SNPs related to prostate cancer, said composite value representing the risk of developing prostate cancer, wherein the composite value is formed from at least 45 SNPs;
d) comparing the SNP composite value to a pre-determined cut-off value established through analysis of cohort data from subjects with prostate cancer; and
e) classifying the individual as being at low risk of prostate cancer according to one or more of the following criteria:
   i. the age of the individual is 80 years or greater;
   ii. the total PSA value is less than 1 ng/ml and the SNP composite value is lower than the predetermined cut-off value;
f) recommending that the individual should have a further prostate cancer diagnostic test within a second time period if one or both of the criteria in step (e) are satisfied.

In the method of the second aspect, individuals are categorised as being at low risk of prostate cancer if they have a total PSA of 1 ng/ml or less and:
The age of the individual is 80 years or greater; and/or
They have an SNP based composite value of less than the pre-determined cut-off value, which may be 1.0 in some embodiments, or 0.784 in some other embodiments.

If either or both of these criteria are satisfied, then the individual is recommended a further prostate cancer diagnostic test within a second time period as defined herein.

In some embodiments of the second aspect, the individual has a total PSA concentration of 1 ng/ml or less but does not meet either of the criteria of step (e) and therefore is not categorised as low risk.

In these embodiments, the method may further comprise classifying the individual as being of moderately low risk of prostate cancer if the individual does not satisfy any of the criteria of step (e) of the second aspect of the invention, and recommending that such individuals should have a further prostate cancer diagnostic test within a third time period as defined herein.

In this way, the method of the second aspect ensures that any individual with a total PSA concentration of 1 ng/ml or less is either categorised as low risk (and a further test is recommended within the second time period) or moderately low risk (and a further test is recommended within the third time period).

As discussed above, a third aspect of the invention is a method for determining an appropriate time interval for further diagnostic testing for prostate cancer in an individual having a total blood prostate-specific antigen (PSA) concentration (measured in unfractionated blood, serum or plasma, particularly serum or plasma) of less than 1 ng/ml, the method comprising the following steps:
a) providing at least one biological sample from the individual;
b) in said biological sample(s) analysing:
   a. the concentration of total PSA; and/or
   b. a category of SNPs related to prostate cancer (SNPpc) by measuring the presence or absence of one or two risk allele(s) of each of a plurality of SNPpc of said category of SNPpc;
c) determining:
   c. the total PSA concentration; and
   d. a composite value based on a combination of the data regarding the SNPs related to prostate cancer, said composite value representing the risk of developing prostate cancer, wherein the composite value is formed from at least 45 SNPs;
d) comparing the SNP composite value to a pre-determined cut-off value established through analysis of cohort data from subjects with prostate cancer; and
e) classifying the individual as being at moderately low risk of prostate cancer according to the following criteria:
   i. the total PSA value is less than 1 ng/ml and the SNP composite value is higher than the predetermined cut-off value;
f) recommending that the individual should have a further prostate cancer diagnostic test within a third time period if the criteria in step (e) is satisfied.

In this aspect of the invention, the individual is categorised as being a moderately low risk of prostate cancer if they have a total PSA concentration of 1 ng/ml or less but do not fulfil the criteria to be categorised a low risk according to the second aspect of the invention. Instead, according to the third aspect of the invention, they are categorised as being moderately low risk of prostate cancer if they have:
A total PSA concentration of 1 ng/ml or less; and
An SNP composite value greater than or equal to the pre-determined cut off value, which may be 1.0 in some embodiments, or 0.784 in some other embodiments as described herein.

In some embodiments of all of the aspects of the present invention, the individual will have a total PSA value of less than 2 ng/ml or less than 1 ng/ml but will not fulfil any of the criteria of step (e) of either the first, second or third aspects described herein. In this case, the individual will be categorised as “moderately low risk” as described herein, and their time to the next diagnostic test will be determined accordingly.

In a further aspect of the invention, there is provided an assay device for performing the methods of the present invention. Therefore, in some embodiments, the invention provides the methods of either the first, second, or third aspects performed using the assay device described herein.

Said assay device may comprise a solid phase having immobilised thereon of ligands, wherein:
the first category of said ligands binds specifically to a defined amount of PSA biomarker, and
the second category of said ligands binds specifically to a defined amount of SNP(s) related to prostate cancer as defined herein, and includes a plurality of different ligands binding specifically to each of said SNPs.

There is also provided a test kit comprising an assay device as defined herein, said test kit further comprising one or more detection molecules for specifically detecting the amount of total and/or free PSA, the SNP(s) related to prostate cancer bound to said first and second category of ligands, respectively. Therefore, in some embodiments, the invention provides the methods of either the first, second, or third aspects performed using the kit described herein.

Typically, one or more of the method steps as described herein, are provided by means of computer software. In particular, there is also provided herein a computer program product directly loadable into the internal memory of a digital computer, wherein the computer program product comprises software code means for at least performing the steps relating to combining data from said individual regarding said PSA concentration, and data from said individual regarding the presence or absence of SNPs related to prostate cancer risk by comparing the PSA concentration (or calculated PSA quotient) or composite value to a pre-determined cut-off value established with cohort data of known prostate cancer and healthy individuals, respectively, for determining the time to the next prostate cancer diagnostic test.

The software implemented aspects of the present invention may be performed locally with respect to the sample analysis within a laboratory, or may instead be performed at a remote location, for example at a remote server that hosts the software. Various known measures may be employed to maintain the security of personal data and the integrity of the source code. In preferred embodiments, the software is an embedded package that connects seamlessly into existing work flows for reporting the results of laboratory analysis to receive the necessary data to perform further data analysis and provide an output.

Preferred, non-limiting examples which embody certain aspects of the invention will now be described, with reference to the following figures and examples:

### DESCRIPTION OF THE FIGURES

FIG. 1:

Genetic Score. The relative occurrence of PC (Gleason≤7) for different ranges of genetic score are shown. The range of each genetic score category is indicated on the x-axis within square brackets. Each category is based on 800 results. Four results were excluded.

FIG. 2:

Genetic Score Reduction. The genetic scores computed on a reduced genotype (50 SNPs) or on the production genotype (101 SNP) are shown (dots). The linear best fit with intercept set to zero is shown as black dots.

FIG. 3:

Projected results from the PCPT equation. The results from the PCPT calculator for total PSA concentration in ng/ml (x-axis) are shown for ages 55, 65 and 75 years (left to right). Results for subjects without previous negative biopsy and family history of PC (those most at risk) are shown as solid thick black lines. Results for individuals with previous negative biopsy and no family history of PC (those least at risk) are shown as thin dashed lines. Intermediate risk categories are shown as solid thin black or thick dashed lines.

FIG. 4:

Projections of individuals with 3% risk for PC from the PCPT calculator. The critical total PSA concentration (y-axis), i.e. resulting in 3% risk, for age categories (x-axis) are shown for a known family history of PC (white column) and for subjects with no known family history of PCs (shaded column).

FIG. 5:

Schematic representation of a classification system as described herein.

### Example 1

The EAU states that “men at elevated risk of having prostate cancer (PC) are those >50 years of age or >45 years with a family history of PC (either paternal or maternal), or African-Americans” may benefit from early detection methods for PC [2].

Age is a significant factor in the risk of having PC [1]. However, with old age the need for diagnosing PC lessens as the expected life time shortens compared to the time it takes for the cancer to progress into a life-threatening disease. The Swedish guidelines and the EAU claim that there is no motivation for testing for PC in individuals with less than 15 years of life expectancy [2, 9], and that Swedish males aged 75 have a life expectancy of 12 years [9].

As the basis for classification described herein, four methods are combined herein into one evaluation that is capable of determining the most appropriate time until a further prostate cancer diagnostic test: the total PSA concentration, the quotient formed by free PSA over total PSA concentration, and two advanced multi-parametric risk calculators: the PCPT and Genetic Score algorithms.

### 1. Total PSA Concentration

The EAU states that “the use of PSA as a serum marker has revolutionised PC diagnosis” and that “as an independent variable, PSA is a better predictor of cancer than either digital rectal examination (DRE) or transrectal ultrasound (TRUS)” [2].

However, concerns are raised that “there are no agreed standards defined for measuring PSA” and that “PSA is organ- but not cancer-specific; therefore, it may be elevated in benign prostatic hypertrophy (BPH), prostatitis and other non-malignant conditions” [2].

As EAU guidelines concerning the early detection for PC based on PSA suggest, “a risk-adapted strategy might be a consideration, based on the initial PSA level” [2].

The risk of PC (ISUP ≥2, which is equivalent to a Gleason score of 7 or greater) when the total PSA concentration is <1 ng/ml is estimated to be between 0.8-1%, and around 2% for total PSA concentrations between 1.1-2 ng/ml [2].

The EAU suggests that testing “could be every two years for those initially at risk, or postponed up to eight to ten years in those not at risk with an initial PSA <1 ng/ml at 40 years and a PSA <2 ng/ml at 60 years of age and a negative family history” [2]. This is also in line with the conclusions of Stattin et al. that claim that “screening can stop in men with PSA below the median (<1 ng/ml) at age 60” [7].

Further, when analysing the data collected in Grönberg et al., 1% of the cohort had high-grade PC (defined as Gleason ≥7) for subjects with total PSA concentration in the interval 0-1 ng/ml. For subjects with total PSA in the interval 1-2 ng/ml, this frequency raised to 2.6% [8].

The current Stockholm3 Laboratory developed test (LDT) by A23 Lab AB associates a risk of 3% for having PC with individuals with a total PSA concentration <1.5 ng/ml and recommends that the patient takes the test in 6-10 years again.

This is also consistent with the findings of Palsdottir et al. that conclude that “men with (pre-index) PSA≤1 ng/ml (45% of men aged 50-74 years) have very low risk to be diagnosed with GS ≥7 cancer at their next PSA test irrespective of testing interval, supporting previous long-term forecasts of risk based on PSA” [3].

Pálsdóttir et al. continue to state that “for men with PSA >1 ng/ml, we observed an increased risk of being diagnosed with GS ≥7 PC with longer than annual testing intervals” followed by noting that “this benefit needs to be balanced against the markedly increased risks for false-positive biopsy recommendations with shorter testing intervals recommendations” [3].

Concerning screening and individuals with total PSA concentration of 1-1.5 ng/ml, Stattin concludes that “for men in their fifties, screening could focus mainly on those in the top decile of PSA (>1.9 ng/ml) because close to half of the subsequent cases of distant metastasis are found in this group” and that “men with lower PSAs should still be screened but less intensively” [7].

Pálsdóttir et al. note that “although the risk of GS ≥7 diagnosis increases with longer testing intervals for men with (pre-index) PSA >1 ng/ml, the absolute increase in risk for men with (preindex) PSA level in the range 1-3 ng/ml is still small (only 651 of 67729 men with (pre-index) PSA level 1-3 ng/ml were diagnosed with GS ≥7 at the next PSA test)” [3].

The EAU states that “men with a PSA >1 ng/ml at 40 years and >2 ng/ml at 60 years are also at increased risk of PC metastasis or death from PC several decades later” [2].

The lowest total PSA concentration discussed in the Swedish care guidelines related to considering other examinations is 2 ng/ml [9].

### 2. Quotient of Free PSA to Total PSA

The quotient free PSA/total PSA (f/t PSA) is considered an indicator of risk for PC, and thus the Swedish care guidelines recommend that the quotient should be considered, along with other factors, when deciding if a patient should have a biopsy performed. It continues to state that the general risk of having PC increases as the quotient decreases. It is estimated that an individual with a quotient >0.25 suffers a risk of 10% and a quotient <0.1 results in a risk of 50% [9]. Here, the discussion is held for individuals with total PSA concentration ≥2 ng/ml [9].

The EAU reports similarly, albeit conditional for individuals with higher total PSA concentrations, that “prostate cancer was detected in men with a PSA 4-10 ng/ml by biopsy in 56% of men with f/t PSA <0.10, but in only 8% with f/t PSA >0.25”.

The relevance of the quotient is confirmed within the data of Grönberg et al. [8]. The authors included free PSA measurements from individuals with a corresponding total PSA concentration >1 ng/ml in the study. The relationship between average PC risk and f/t PSA quotient in this data set was analysed in order to identify a quotient level for which men with a quotient below said level had sufficiently high risk to be recommended a new test with higher recurrence.

Out of the first 4004 consecutive data points in the original study by Grönberg et al. [8], 361 had PSA values between 1 and 2 ng/ml. Of these, 26 (7%) had cancer of Gleason ≥7. Among the 100 patients with quotient <0.11, nine (9%) cancers were found. Among the remaining patients with quotient >0.11, 6% had cancer. When looking at the calculated risk score (albeit using a slightly different version than the currently used equation), the subjects with small quotient had in average 20.6% estimated risk, and the remaining, 14.5% risk.

Further, these historic results were compared to those currently available from the Stockholm3 LDT (A23 Lab AB). A total of 600 cases with PSA values in the range of 1.5-2 ng/ml (measured by A23 Lab AB using a Thermo Fisher Kryptor device and plasma samples) were analysed in terms of risk conditional their quotient values. The average estimated risk in this cohort is 7.8%, which is in line with the actual observation of cancer frequency in the original study [8]. Amongst these, 31 individuals with quotients below 0.11 were found. The average risk was estimated to be 18%, whereas the remaining ones had an estimated average risk of 7.2%.

A technical issue is that the commonly used assays for free PSA have a sensitivity down to approximately 0.05 ng/ml. This means that it is meaningless to measure free PSA for total PSA values around 1 ng/ml, because the quotient would suffer from the lack of accuracy and precision of an assay operating close to the limit of its sensitivity.

The EAU also urges caution in the use of the quotient on technical grounds stating that “f/t PSA must be used cautiously because it may be adversely affected by several pre-analytical and clinical factors (e.g., instability of free PSA at 4ºC and room temperature, variable assay characteristics, and concomitant BPH in large prostates)” [2].

### 3. Genetic Score

Family history and genetic profile are considered factors that influence the risk of PC [8]. In Grönberg et al. the authors investigated the contribution of several Single Nucleotide Polymorphisms (SNPs) to the risk of having PC in males aged 50-69 years [8]. A Genetic Score was defined, which is integrated in an extended risk estimator that is the basis for the Stockholm3 test.

In Stockholm3 LDT (A23 Lab AB), a large panel of SNP markers are typed for each subject with PSA above the current reflex cut-off, 1.5 ng/ml. The SNPs are germline and therefore the genotype and the Genetic Score of an individual is fixed throughout life [8]. Not all aspects of the kinetics of disease development with respect to genetics are known. However, there could be groups of individuals with greater propensity for PC based on their genetic score that may benefit from the recommendation of undergoing a new test sooner. In their study, Grönberg et al. note that “men in the top decile of the genetic score have a 25% risk of cancer with a Gleason score of at least 7” [8].

Further, the authors discuss the outlook of individualization of PC testing using genetic profiling, suggesting that “it is inexpensive to measure (and the price is constantly dropping), it only needs to be measured once in a man's lifetime, and it is important for men with a very high genetic risk” [8].

The relation between the genetic score and occurrence of Gleason score >7 cancer was assessed by revisiting the first 4004 consecutive data points from the original study by Grönberg et al. [8]. In the study, subjects with total PSA concentration ≥1 ng/ml were included.

The genetic score is defined as an accumulation of risks associated with each contributing SNP. Here, all contributions are considered as being independent, and the removal of any number of SNPs from the calculation will therefore result in a genetic score in the same range and with the same interpretation. For Stockholm3, the current protocols allow omitting maximally three individual SNP results. In the original study by Grönberg et al. [8], 10-15 SNPs per patient were sometimes omitted for technical reasons. Hence the genetic score output is comparable irrespective of which generation has been used, however the thresholds may be adjusted slightly depending on which version is used.

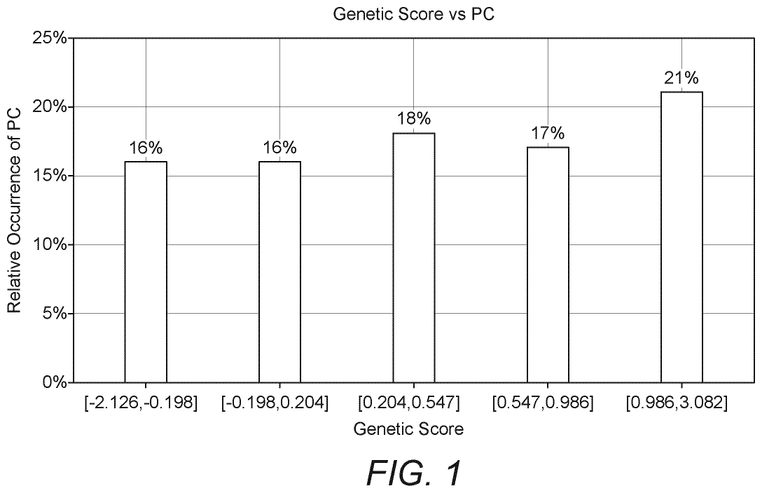

A genetic score greater than 0.986 results in an increase of cancer findings from 16-18% to 21% (see FIG. 1). Since the risk for PC due to the subjects' genotype is invariant over time, individuals with a genetic score greater than 0.986 should add about 3 units to the risk estimated using biomarkers, age and the similar. Further, since the average risk for prostate cancer is around 1% for individuals with a total PSA concentration of up to 1 ng/ml [2] and around 2-3% for total PSA concentrations of 1-2 ng/ml, the subpopulation with a higher genetic score would have a 4-6% risk (by adding 3 units).

To facilitate the workflow, the original panel of SNPs used by Grönberg et al. was reduced in the Stockholm3 LDT test (A23 Lab AB), and the least contributing markers of the original method were removed after confirming that the effect caused by removing them was negligible. Therefore, the genetic score used by Grönberg and colleagues was more inclusive than the one used in current practice. Herein, a further reduction for this framework is suggested. To this end, the effects of using the 50 most prominent contributing SNPs compared to using the current panel was investigated.

The 50 SNPs used in the methods described herein are:
rs138213197; rs7818556; rs6983267; rs10993994; rs12793759; rs16901979; rs9911515; rs1016343; rs7106762; rs6579002; rs16860513; rs5945619; rs16902094; rs10896437; rs651164; rs7679673; rs13265330; rs2047408; rs10107982; rs620861; rs9297746; rs1992833; rs7213769; rs2710647; rs888507; rs17021918; rs12500426; rs2028900; rs7102758; rs16901922; rs6062509; rs2659051; rs12543663; rs4699312; rs11091768; rs3120137; rs6794467; rs10086908; rs2315654; rs12151618; rs747745; rs1009; rs2132276; rs2735839; rs11568818; rs684232; rs9364554; rs2660753; rs10807843; rs1933488.

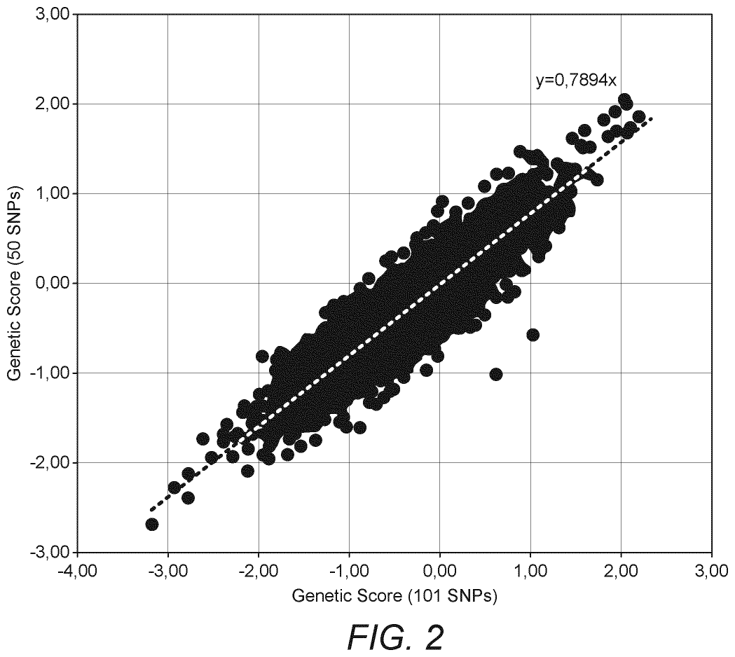

The comparison was made for the same data set of 4000 individuals included in the original study by Grönberg et al. [8]. We found that the genetic score based on the reduced input set is highly consistent with that corresponding to the extended, production set of SNPs (see FIG. 2). It therefore appears that the genetic score based on 50 SNPs has an adequate capacity to distinguish individuals with elevated genetic risk.

### 4. PCPT

The Prostate Cancer Prevention Trial (PCPT) conducted in the US is regarded as one high-quality clinical study of prostate cancer. The PCPT integrates several factors as inputs and it published a risk score equation [10].

The equation used for the PCPT risk score is as follows (source code from MATLAB):

$$function p = a 23 \_pcpt \_calc ( psa , famhist , prevbiopsy , age , dre )$$

$$race = 0 ;$$

$$y = - 6.25 + 1.29 * log ( psa ) + 0.27 * famhist + 1 * dre - 0.36 * prevbiopsy + 0.03 * age + 0.96 * race ;$$

$$or = exp ( y ) ;$$

$$p = or / ( 1 + or ) * 100 ;$$

The EAU states that “risk calculators may be useful in helping to determine (on an individual basis) what the potential risk of cancer may be, thereby reducing the number of unnecessary biopsies”. Furthermore, it explicitly lists the PCPT among other leading calculators.

Herein, the PCPT equation for calculating the risk of having Gleason >7 is suggested. The risk PCPT calculator accepts input related to finding from a digital rectal exam, DRE, and ethnicity that are not easily accommodated within the framework described herein. Herein it will be assumed that the subjects have not undergone a DRE or have had the procedure performed but with no disease findings. As this may not be true for all subjects, consequently in some cases the test may underestimate of the risk for some patients of having prostate cancer. Regarding ethnicity in the PCPT equation, if the patient perceives himself as being Afro-American, this is mathematically represented as a one or a zero [10]. It is difficult to predict how these categories translate to other populations and therefore within the present study, which did not study the American market, the ethnicity coefficient will be also be set to zero as this is what the majority of subjects in the original study answered no to the aforementioned question. As research into how ethnicity affects the risk of having prostate cancer progresses, updates to the model may be made accordingly.

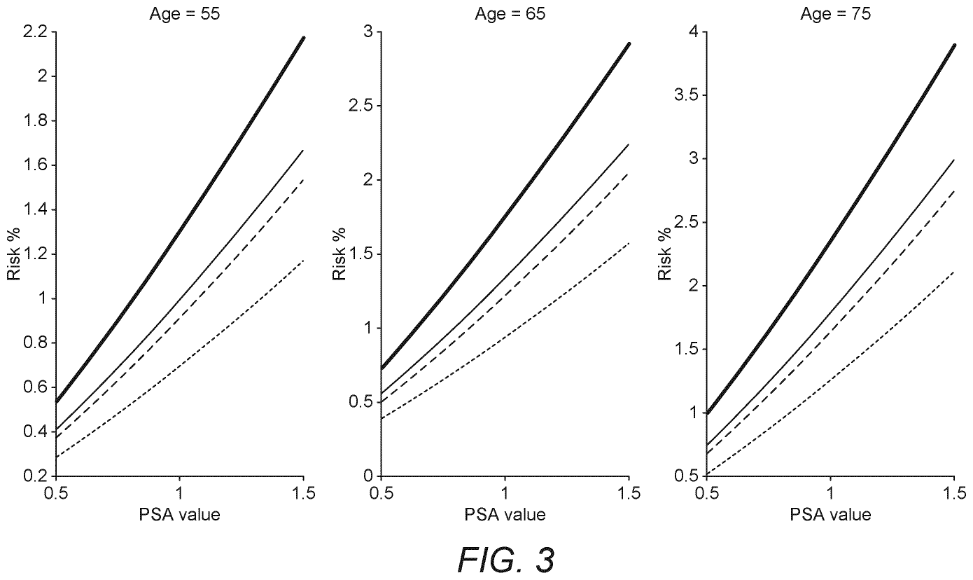

In FIG. 3, results from the calculator are shown for combinations of age, previous biopsy and total PSA concentration.

We found that for subjects herein, the PCPT estimated risk levels are generally <3%. Only for those aged >65 with a family history of prostate cancer and that have not undergone a previous negative biopsy the PC risk of Gleason score ≥7 will be >3% (see FIG. 3, middle and right, solid dark line). A risk >3% should be considered “normal” and the recommendation should be to conduct a new test in 2 years.

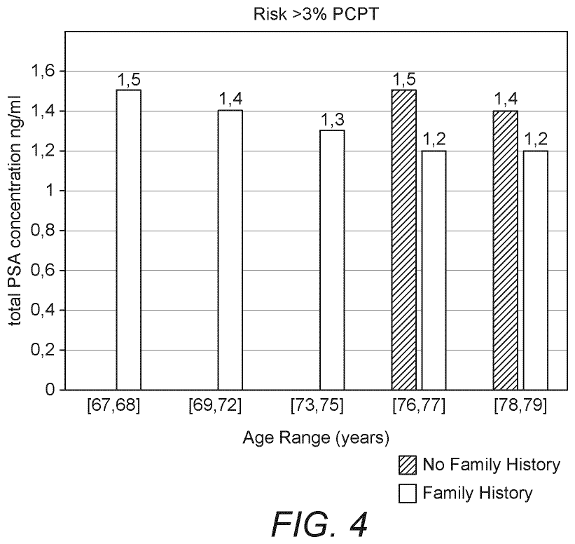

Thus, the PCPT risk score calculator can only identify a small fraction of elderly individuals with risk exceeding 3%. In more detail, the following combinations will result in risk >3% (see FIG. 4).

### 5. Categorisation

Three recurrence categories are suggested for the test defined herein based on recommendations discussed previously. As the names indicates, the categories are intended to be continuous representations of risk, conceptually escalating in the order GREEN (low risk), LIME (moderately low risk) and YELLOW (normal risk). Here we present the classification criteria.

### 5.1 GREEN (Low Risk)

For the lowest level of recommended recurrence in testing, GREEN, the longest interval, 6-10 years, will be recommended. Before retaking the test, the multiparametric elevated PSA test is recommended to the care provider.

The recurrence status of an individual will be classified as GREEN if any of the following apply:
Total PSA concentration is less than 1 ng/ml and Genetic Score is less than 1 (equivalent to using 101 SNPs, corresponding to a cut off value of 0.784 when using 45-50 SNPs);
The age of the individual is ≥80 years.

The recommendation to the care provider will state “Low risk for prostate cancer, test again in 6-10 years. Risk=1%”. The second condition is motivated by the guidelines of the EUA.

### 5.2 YELLOW (Normal Risk)

The category YELLOW is the most severe recurrence category, where all subjects have a total PSA value greater than or equal to 1 ng/ml.

The recurrence status of an individual is YELLOW if any of the following applies:
The quotient f/t PSA <0.11, and the total PSA ≥1.3 ng/ml;
The risk from PCPT is ≥3%;
The genetic score is greater than 1 (equivalent to using 101 SNPs, corresponding to a cut off value of 0.784 when using 45-50 SNPs).

The recommendation to the care provider will state “Normal risk for prostate cancer, test again in 2-4 years. Risk=4%”.

### 5.3 LIME (Moderately Low Risk)

The category LIME is an intermediate recurrence category and is simply defined as the complement to GREEN and YELLOW, i.e. if a:
Recurrence result is neither GREEN nor YELLOW, it is LIME.

The intended recommendation will be “Moderately low risk for prostate cancer, test again in 4-6 years. Risk=3%”.

5.4 Recommendation to Proceed with an Advanced Test

Finally, if the total PSA concentration of an individual is greater than 1.5 ng/ml, the recommendation will be “An additional test is recommended, such as Stockholm3, 4K, PHI or EPI.

There will also be an error category, in the event that test could not be performed given the available resources.

### 6. Conclusions

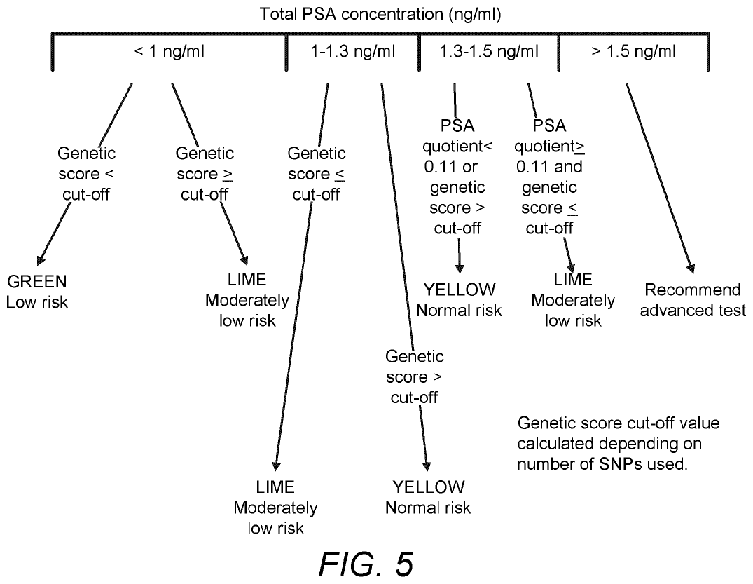

In conclusion, the ability to individualise statements for men with total PSA concentrations of less than 2 ng/ml has been evaluated. Four methods assessing the eligibility for three distinct testing category intervals, or a recommendation to the subject to proceed to a more advanced test, are the basis for the test described herein. This is summarised schematically in FIG. 5.

Taken together, there is circumstantial evidence that a quotient of f/t PSA <0.11 is indicative of higher risk for PC, also for PSA values near but below 2 ng/ml. There is evidence that the overall risk for individuals with PSA <1 ng/ml is very low (1% according to PCPT) and technical arguments for not measuring free PSA for individuals with PSA <1 ng/ml. Hence, it is justified to assign individuals with PSA between 1 and 2 ng/ml, and with a quotient less than 0.11, a “normal risk” indication and recommend a new test in 2-4 years. An estimated 5% of men with PSA values between 1 and 2 ng/ml would get a “normal risk” value due to this reasoning.

Analysis of the quotient and using the PCPT model are simple from a lab point of view, though only a small fraction of individuals will be upgraded to “normal risk”. Including the genetic score as basis for upgrading will add another approximately 10% of the low PSA population to the “normal risk” category.

The inclusion of the PCPT model is not essential and therefore may not be implemented (clinical benefit to technical risk is moderate). In some cases, a look-up table based on the equation may be a safer mode of implementation.

A risk level >3% should result in a recommendation to conduct a new test within 2-4 years. Hence, this provides evidence that individuals with genetic score >1 (equivalent to) should be recommended to repeat tests in 2-4 years. This would cover at the most 20% of the tested population.

Pálsdóttir et al. [3] state that “tailored testing intervals (should) be part of a systematised and individualised pipeline for prostate cancer diagnostics to reduce unintended consequences of testing and lower prostate cancer mortality”, which is the intended outcome of the test described herein.

### Example 2

This example illustrates how the method works in comparison to current clinical practice using a data set from Sweden. The dataset consists of data collected in Stockholm County from 15170 participants of the STHLM3 study [8], where data was extracted from either the study itself (2013-2015) or from an entry in the STHLM0 database (2003-2020) [3]. Current clinical practice was defined as the 2018 clinical guidelines for prostate cancer in Sweden [https://cancercentrum.se].

A few elements of data were missing in the Stockholm County cohort. As a representative for a normal cohort, about 500 individuals from Stavanger in Norway were analysed with respect to how the three categories disclosed herein (green, lime, and yellow) were distributed. This and similar distributions were applied to the Stockholm County cohort.

Data from the Stockholm County cohort contains results from multiple PSA tests taken over time, in some cases PSA values over a decade per individual. It therefore becomes possible to determine if a statement at a first time point was reasonable, because there is a PSA test at a later second time point to confirm or reject the adequacy of the statement made at the first time point.

When applying the method to the subjects in the cohort and comparing the results from the method to the output of clinical practice, it is clear that the use of the method disclosed herein will improve the quality of care for patients compared to current clinical practice.

If testing of eligible individuals would be performed using the method disclosed herein instead of according to the recommendations currently used to guide clinical practice the health risks to patients are expected to decrease as the method is estimated to:
decrease the cumulative risk of suffering unnecessary biopsies by approximately 60%,
decrease the testing frequency by approximately 45%.

This is in addition to performing at least as well at predicting clinically significant prostate cancer as using the current gold standard, total PSA.

### REFERENCES

[1] European Association of Urology, Uroweb PROSTATE CANCER EPIDEMIOLOGY AND AETIOLOGY, 2020. [Online]. Available: https://uroweb.org/guideline/prostate-cancer/#3.
[2] European Association of Urology, Uroweb PROSTATE CANCER DIAGNOSTIC EVALUATION, 2020. [Online]. Available: https://uroweb.org/guideline/prostate-cancer/#5.
[3] T. Palsdottir et al., The impact of different prostate-specific antigen (PSA) testing intervals on Gleason score at diagnosis and the risk of experiencing false-positive biopsy recommendations: a population based cohort study, BMJ Open, p. doi: 10.1136/,2019.
[4] Eggener S E, et al., Gleason 6 prostate cancer: translating biology into population health, J Urol, 2015.
[5] Ahmed H U et al., Do low-grade and low-volume prostate cancers bear the hallmarks of malignancy?, Lancet Oncol, 2012.
[6] Loeb S, et al., Overdiagnosis and overtreatment of prostate cancer, Eur Urol, 2014.
[7] Stattin, P et al., Improving the Specificity of Screening for Lethal Prostate Cancer, European Urology, 2015.
[8] Grönberg H. et al., “Prostate cancer screening in men aged 50-69 years,” Lancet Oncol, 2015.
[9] Regionala cancercentrum i samverkan, “Prostatacancer Nationell Vårdguide,” 2020. [Online]. Available: https://www.cancercentrum.se/globalassets/cancerdiagnoser/prostatacancer/vardpro gram/nationelltvardprogram-prostatacancer.pdf.
[10] Thompson et al., “Assessing Prostate Cancer Risk: Results from the Prostate,” Journal of the National Cancer Institute, pp. 529-534, 2006.
[11] Schmid et al., “Observations on the doubling time of prostate cancer. The use of serial prostate-specific antigen in patients with untreated disease as a measure of increasing cancer volume,” Cancer., p. 2031-40, 1993.
[12] Egawa et al., “Impact of Life Expectancy and Tumor Doubling Time on the Clinical Significance of Prostate Cancer in Japan,” Jpn J Clin Oncol, p. 394-400, 1997.

1. A method for determining an appropriate time interval for further diagnostic testing for prostate cancer in an individual having a total blood prostate-specific antigen (PSA) concentration, measured in unfractionated blood, serum or plasma, of less than or equal to 2 ng/ml, the method comprising the following steps:
a) providing at least one biological sample from the individual;
b) in said biological sample(s) analysing:
i. the concentration of free PSA and total PSA; and/or
ii. a category of SNPs related to prostate cancer (SNPpc) by measuring the presence or absence of one or two risk allele(s) of each of a plurality of SNPpc of said category of SNPpc;
c) determining at least one of:
c. the quotient of free PSA/total PSA; and
d. a composite value based on a combination of the data regarding the SNPs related to prostate cancer, said composite value representing the risk of developing prostate cancer wherein the composite value is formed from at least 45 SNPs;
d) comparing the quotient of free PSA/total PSA to a pre-determined cut-off value established through analysis of cohort data from subjects with prostate cancer with known free PSA and total PSA; and/or comparing the SNP composite value to a pre-determined cut-off value established through analysis of cohort data from subjects with prostate cancer; and
e) classifying the individual as being at normal risk of prostate cancer according to one or more of the following criteria:
i. the PSA quotient (free PSA (ng/ml)/total PSA (ng/ml)) is equal to or less than the pre-determined cut off value and the total PSA is from 1.3 to 1.5 ng/ml;
ii. the SNP composite value is greater than the pre-determined cut off value and the total PSA value is greater than or equal to 1 ng/ml;
f) recommending that the individual should have a further prostate cancer diagnostic test within a first time period if one or both of the criteria in step (e) are satisfied.

2. The method of claim 1, wherein:
i. step b) further comprises assessing the individual's risk of having prostate cancer using the Prostate Cancer Prevention Trial (PCPT) equation;
ii. step d) further comprises comparing the risk of the individual having prostate cancer calculated using the PCPT equation to a predetermined reference value, representing the risk of developing prostate cancer;
iii. step e) further comprises classifying the individual as normal risk of prostate cancer if the PCPT equation score is above or equal to the reference value.

3. The method of claim 1 or claim 2, wherein step c) further comprises determining the age of the individual, and step e) further comprises classifying the individual as being of low risk of prostate cancer if one or more of the following applies:
iii) the age of the individual is 80 years or greater;
iv) the total PSA value is less than 1 ng/ml and the SNP composite value is lower than the predetermined cut-off value;
and step f) further comprises recommending that such individuals should have a further prostate cancer diagnostic test within a second time period.

4. The method of claim 1 or 2, wherein the method further comprises classifying the individual as being of moderately low risk of prostate cancer if the individual does not satisfy any of the criteria of step (e), and recommending that such individuals should have a further prostate cancer diagnostic test within a third time period.

5. The method of any one of claims 2-4, wherein the PCPT equation includes at least the following inputs: age of the individual; total PSA concentration (ng/ml); whether there is a family history of prostate cancer; and whether the individual has previously had a negative prostate cancer biopsy.

6. The method of claim 5, wherein the PCPT equation also includes the input of the race of the individual, optionally wherein the individual is categorised is one of the following: White; African American; or Other.

7. The method of claims 2-6, wherein the PCPT equation is used to calculate the risk of the individual having prostate cancer with a Gleason Score of 7 or above.

8. The method of any one of claims 2-7, wherein the predetermined reference value for the risk of the individual having prostate cancer with a Gleason Score of 7 or above calculated with the PCPT equation is 3%.

9. The method of any of claims 1-8, wherein an advanced prostate cancer diagnostic test is recommended if the individual has a total PSA concentration of greater than 1.5 ng/ml.

10. The method of any one of claims 1-8, wherein the total PSA concentration is 1.5 ng/ml or less.

11. The method of any one of claims 1-10, wherein the pre-determined cut off value of the PSA quotient is from 0.10 to 0.12.

12. The method of any one of claims 1-11, wherein the pre-determined cut off value of the PSA quotient is about 0.11.

13. A method for determining an appropriate time interval for further diagnostic testing for prostate cancer in an individual having a total blood prostate-specific antigen (PSA) concentration, measured in unfractionated blood, serum or plasma, of less than 1 ng/ml, the method comprising the following steps:
a) providing at least one biological sample from the individual;
b) in said biological sample(s) analysing:
a. the concentration of total PSA; and/or
b. a category of SNPs related to prostate cancer (SNPpc) by measuring the presence or absence of one or two risk allele(s) of each of a plurality of SNPpc of said category of SNPpc;
c) determining the age of the individual and/or the following:
c. the total PSA concentration; and
d. a composite value based on a combination of the data regarding the SNPs related to prostate cancer, said composite value representing the risk of developing prostate cancer, wherein the composite value is formed from at least 45 SNPs;
d) comparing the SNP composite value to a pre-determined cut-off value established through analysis of cohort data from subjects with prostate cancer; and
e) classifying the individual as being at low risk of prostate cancer according to one or more of the following criteria:
i. the age of the individual is 80 years or greater;
ii. the total PSA value is less than 1 ng/ml and the SNP composite value is lower than the predetermined cut-off value;
f) recommending that the individual should have a further prostate cancer diagnostic test within a second time period if one or both of the criteria in step (e) are satisfied.

14. The method of claim 13, wherein the method further comprises classifying the individual as being of moderately low risk of prostate cancer if the individual does not satisfy any of the criteria of step (e), and recommending that such individuals should have a further prostate cancer diagnostic test within a third time period.

15. A method for determining an appropriate time interval for further diagnostic testing for prostate cancer in an individual having a total blood prostate-specific antigen (PSA) concentration, measured in unfractionated blood, serum or plasma, of less than 1 ng/ml, the method comprising the following steps:
a) providing at least one biological sample from the individual;
b) in said biological sample(s) analysing:
a. the concentration of total PSA; and/or
b. a category of SNPs related to prostate cancer (SNPpc) by measuring the presence or absence of one or two risk allele(s) of each of a plurality of SNPpc of said category of SNPpc;
c) determining:
i. the total PSA concentration; and
ii. a composite value based on a combination of the data regarding the SNPs related to prostate cancer, said composite value representing the risk of developing prostate cancer, wherein the composite value is formed from at least 45 SNPs;
d) comparing the SNP composite value to a pre-determined cut-off value established through analysis of cohort data from subjects with prostate cancer; and
e) classifying the individual as being at moderately low risk of prostate cancer according to the following criteria:
i. the total PSA value is less than 1 ng/ml and the SNP composite value is higher than the predetermined cut-off value;
f) recommending that the individual should have a further prostate cancer diagnostic test within a third time period if the criteria in step (e) is satisfied.

16. The method of any one of claims 1-15, wherein the further prostate cancer diagnostic test is an advanced prostate cancer diagnostic test.

17. The method of claim 16, wherein the advanced prostate cancer diagnostic test has a false positive rate of less than or equal to 40%.

18. The method of claim 16 or 17, wherein the advanced prostate cancer diagnostic test is selected from the group consisting of: Stockholm 3; 4K; Phi; and EPI.

19. The method of any one of claims 1-18 wherein the biological sample is an unfractionated blood sample or a serum sample or a plasma sample, optionally wherein the biological sample is a blood sample or a serum sample.

20. The method of any one of claims 1-19 wherein the biological sample for measurement of the concentration of total PSA and/or free PSA is a serum sample or a plasma sample.

21. The method of any one of claims 1-20 wherein the biological sample for measuring the presence or absence of one or two risk allele(s) of each of a plurality of SNPpc is an unfractionated blood sample or a saliva sample.

22. The method of any one of claim 1, 2, 5-12 or 16-21 wherein the first time period is from about 2 years to about 4 years.

23. The method of any one of claim 3 or 5-13, wherein the second time period is from about 6 years to about 10 years.

24. The method of any one of claim 4-12 or 14-21, wherein the third time period is from about 4 years to about 6 years.

25. The method of any one of claims 1-24, wherein the SNP composite value is determined according to a pre-determined equation.

26. The method of any one of claims 1-25, wherein the measurement of the presence or absence of each of the plurality of SNPpc is conducted by MALDI mass spectrometry or a PCR-based SNP genotyping assay (e.g. the TaqMan assay).

27. The method of any one of claims 1-26, wherein the category of SNPpc include at least 45 SNPs selected from the group consisting of: rs138213197; rs7818556; rs6983267; rs10993994; rs12793759; rs16901979; rs9911515; rs1016343; rs7106762; rs6579002; rs16860513; rs5945619; rs16902094; rs10896437; rs651164; rs7679673; rs13265330; rs2047408; rs10107982; rs620861; rs9297746; rs1992833; rs7213769; rs2710647; rs888507; rs17021918; rs12500426; rs2028900; rs7102758; rs16901922; rs6062509; rs2659051; rs12543663; rs4699312; rs11091768; rs3120137; rs6794467; rs10086908; rs2315654; rs12151618; rs747745; rs1009; rs2132276; rs2735839; rs11568818; rs684232; rs9364554; rs2660753; rs10807843; rs1933488.

28. The method of any one of claims 1-26 wherein the category of SNPpc include all 50 SNPs of claim 27.

29. The method of any one of claims 27-28, wherein the category of SNPpc include measuring at least further one SNPpc selected from the group consisting of: rs17467139; rs12947919; rs2331780; rs1894292; rs2107131; rs6545962; rs11649743; rs758643; rs2297434; rs902774; rs17224342; rs5918762; rs17138478; rs3019779; rs1873555; rs12946864; rs12475433; rs3765065; rs4871779; rs10875943; rs11601037; rs6489721; rs11168936; rs9297756; rs11900952; rs6569371; rs7752029; rs5934705; rs3745233; rs1482679; rs749264; rs6625760; rs5978944; rs2366711; rs5935063; rs10199796; rs2473057; rs4925094; rs3096702; rs12490248; rs4245739; rs10094059; rs306801; rs2823118; rs2025645; rs9359428; rs10178804; rs6090461; rs2270785; rs16901841; rs2465796.

30. The method of any one of claims 27-28, wherein the category of SNPpc include measuring all of the SNPpc selected from the group consisting of: rs17467139; rs12947919; rs2331780; rs1894292; rs2107131; rs6545962; rs11649743; rs758643; rs2297434; rs902774; rs17224342; rs5918762; rs17138478; rs3019779; rs1873555; rs12946864; rs12475433; rs3765065; rs4871779; rs10875943; rs11601037; rs6489721; rs11168936; rs9297756; rs11900952; rs6569371; rs7752029; rs5934705; rs3745233; rs1482679; rs749264; rs6625760; rs5978944; rs2366711; rs5935063; rs10199796; rs2473057; rs4925094; rs3096702; rs12490248; rs4245739; rs10094059; rs306801; rs2823118; rs2025645; rs9359428; rs10178804; rs6090461; rs2270785; rs16901841; rs2465796.

31. The method of any one of claims 1-30, wherein the method allows for disregarding at least about 5% of the SNPpc of the SNPpc category when determining the SNP composite value.

32. A computer implemented method for determining an appropriate time interval for further diagnostic testing for prostate cancer in an individual having a total blood prostate-specific antigen (PSA) concentration, measured in unfractionated blood, serum or plasma, of less than or equal to 2 ng/ml, the method comprising the following steps:
receiving data obtained from analysis of one or more biological samples taken from an individual, the data representing the concentration of free PSA and total PSA present in the sample; and
receiving data for a category of SNPs related to prostate cancer (SNPpc) indicating the presence or absence of one or two risk allele(s) of each of a plurality of SNPpc of said category of SNPpc;
processing the data to determine:
i. the quotient of free PSA/total PSA; and
ii. a composite value based on a combination of the data regarding the SNPs related to prostate cancer, said composite value representing the risk of developing prostate cancer wherein the composite value is formed from at least 45 SNPs;
comparing the quotient of free PSA/total PSA to a pre-determined cut-off value established through analysis of cohort data from subjects with prostate cancer with known free PSA and total PSA; and comparing the SNP composite value to a pre-determined cut-off value established through analysis of cohort data from subjects with prostate cancer; and
classifying the individual as being at normal risk of prostate cancer according to one or more of the following criteria:
i. if the PSA quotient (free PSA (ng/ml)/total PSA (ng/ml)) is equal to or less than the pre-determined cut off value and the total PSA is from 1.3 to 1.5 ng/ml; and
ii. if the SNP composite value is greater than the pre-determined cut off value and the total PSA value is greater than or equal to 1 ng/ml.

33. A computer implemented method for determining an appropriate time interval for further diagnostic testing for prostate cancer in an individual having a total blood prostate-specific antigen (PSA) concentration, measured in unfractionated blood, serum or plasma, of less than 1 ng/ml, the method comprising the following steps:
receiving data obtained from analysis of one or more biological samples taken from an individual of a known age, the data representing the concentration of total PSA present in the sample;
receiving data for a category of SNPs related to prostate cancer (SNPpc) indicating the presence or absence of one or two risk allele(s) of each of a plurality of SNPpc of said category of SNPpc;
processing the data to determine a composite value based on a combination of the data regarding the SNPs related to prostate cancer, said composite value representing the risk of developing prostate cancer wherein the composite value is formed from at least 45 SNPs;
comparing the SNP composite value to a pre-determined cut-off value established through analysis of cohort data from subjects with prostate cancer; and
classifying the individual as being at low risk of prostate cancer according to one or more of the following criteria:
i. the age of the individual is 80 years or greater; and
ii. the SNP composite value is lower than the predetermined cut-off value.

34. A computer implemented method for determining an appropriate time interval for further diagnostic testing for prostate cancer in an individual having a total blood prostate-specific antigen (PSA) concentration, measured in unfractionated blood, serum or plasma, of less than 1 ng/ml, the method comprising the following steps:
receiving data obtained from analysis of one or more biological samples taken from an individual, the data representing the concentration of total PSA present in the sample;
receiving data for a category of SNPs related to prostate cancer (SNPpc) indicating the presence or absence of one or two risk allele(s) of each of a plurality of SNPpc of said category of SNPpc;
processing the data to determine a composite value based on a combination of the data regarding the SNPs related to prostate cancer, said composite value representing the risk of developing prostate cancer wherein the composite value is formed from at least 45 SNPs;
comparing the SNP composite value to a pre-determined cut-off value established through analysis of cohort data from subjects with prostate cancer; and
classifying the individual as being at moderately low risk of prostate cancer if the total PSA value is less than 1 ng/ml and the SNP composite value is higher than the predetermined cut-off value.

35. A computer implemented method for determining an appropriate time interval for further diagnostic testing for prostate cancer in an individual of a known age having a total blood prostate-specific antigen (PSA) concentration, measured in unfractionated blood, serum or plasma, of less than or equal to 2 ng/ml, the method comprising the following steps:
receiving data obtained from analysis of one or more biological samples taken from an individual, the data representing the concentration of free PSA and total PSA present in the sample; and
receiving data for a category of SNPs related to prostate cancer (SNPpc) indicating the presence or absence of one or two risk allele(s) of each of a plurality of SNPpc of said category of SNPpc;
processing the data to determine:
i. the quotient of free PSA/total PSA; and
ii. a composite value based on a combination of the data regarding the SNPs related to prostate cancer, said composite value representing the risk of developing prostate cancer wherein the composite value is formed from at least 45 SNPs;
comparing the quotient of free PSA/total PSA to a pre-determined cut-off value established through analysis of cohort data from subjects with prostate cancer with known free PSA and total PSA;
comparing the SNP composite value to a pre-determined cut-off value established through analysis of cohort data from subjects with prostate cancer;
and,
classifying the individual as being at normal risk of prostate cancer if either:
i. the PSA quotient (free PSA (ng/ml)/total PSA (ng/ml)) is equal to or less than the pre-determined cut off value and the total PSA is from 1.3 to 1.5 ng/ml; or
ii. the SNP composite value is greater than the pre-determined cut off value and the total PSA value is greater than or equal to 1 ng/ml,
or,
classifying the individual as being at low risk of prostate cancer if either:
i. the age of the individual is 80 years or greater; or
ii. the total PSA concentration is less than 1 mg·ml and the SNP composite value is lower than the predetermined cut-off value,
or,
classifying the individual as being at moderately low risk of prostate cancer if the total PSA value is less than 1 ng/ml and the SNP composite value is higher than the predetermined cut-off value.

36. A computer program product storing computer executable instructions for performing the computer implemented steps of the method of any preceding claim.

37. A method, computer implemented method, or computer program product substantially as described herein with reference to the description, examples and figures.
